(19) United States
(12) Patent Application Publication
(10) Pub. No.: US 2005/0153920 A1
(43) Pub. Date: Jul. 14, 2005

(54) Ribozymes used as prodrugs

(75) Inventor: Asher Nathan, Bet Shemesh (IL)

Correspondence Address: BROWDY AND NEIMARK, P.L.L.C.;624 NINTH STREET, NW, SUITE 300, WASHINGTON, DC, 20001-5303, US

(73) Assignee: Intelligene Ltd., Jerusalem (IL)

(21) Appl. No.: 11/000,191

(22) Filed: Dec. 1, 2004

Related U.S. Application Data
(63) Continuation of application No. 09/830,085, filed on Jul. 23, 2001, now abandoned.

(30) Foreign Application Priority Data
Oct. 23, 1998 (IL) 126732

(51) Int. Cl. A61K 48/00; A61K 31/7072; A61K 31/7076

(52) U.S. Cl. 514/44; 514/45; 514/49

(57) ABSTRACT

The invention concerns prodrugs wherein the drug converts from its inactive form to its active form due to catalytic activity of nucleic acid sequences associated with the prodrug.

### RELATED APPLICATIONS

This is a continuation of copending application Ser. No. 09/830,085, nationalized Apr. 23, 2001, which application is the national pahse of INternational applicaiton PCT/IL99/00556, filed, Oct. 22, 1998.

### FIELD OF THE INVENTION

This invention concerns prodrugs and methods for their preparation.

### BACKGROUND OF THE INVENTION

The following are publications believed to be relevant to the invention:

1. Porter, H. and Lizardi, P. M., “An allosteric hammerhead ribozyme”, Bio/Technology, 13:101-104 (1995).

2. WO 94/13833.

3. WO 98/08975.

The term “prodrug” is generally used to denote drugs that are administered to the patient in an inactive form but which become physiologically active in the body of the patient usually due to some sort of enzymatic processing in the body of the patient. The activation may occur, for example, due to proteolytic digestions inside the target cells to which the prodrug entered, or may be due to processing by enzymes in the patient's liver.

Ribozymes are RNA molecules having catalytic activity. Naturally occurring ribozymes are capable of cleavage, with high specificity, of RNA sequences. It has previously been suggested that this enzymatic property of ribozymes may be utilized in order to destroy undesired RNA sequences in the cells for example sequences of HIV viruses.

### SUMMARY OF THE INVENTION

By one aspect termed “the prodrug aspect”, the present invention concerns a prodrug for use in inducing a physiological effect in a target cell population, comprising a drug which can be converted from an inactive form, in which the drug does not induce the physiological effect, to a physiologically active form, in which form the drug induces said physiological effect; the prodrug comprising the drug in the inactive form and being characterized by:

the prodrug comprises a complex between a nucleic acid molecule and said drug in the inactive form; the nucleic acid molecule being capable of transition from an initially catalytically inactive form to a catalytically active form, in which catalytically active form the molecules causes said drug to convert from the inactive form to the active form; said transition of the nucleic acid molecule occurring inside, on the membrane, or in vicinity of cells of said population; the molecule in the prodrug is in the inactive form.

The term “prodrug” concerns a composition which has the potential of producing a desired physiological effect on cells, but is initially inert (i.e. does not produce said effect), and only after undergoing some modifications becomes physiologically active and produces said physiological effect on cells.

The term “physiological effect” concerns any effect a drug may have on cells, in order to improve the health of the subject administered with the drug. The effect is produced in order to treat, prevent a disease, a defect or pathological condition or to alleviate some of the manifestations of a disease, defect or pathological condition. The physiological effect may be a cytotoxic effect wherein diseased cells are destroyed. The physiological effect may also be a decrease in the rate of proliferation of cells (for example, cancer cells, blood vessels surrounding cancer cells, or cells of the immune system, etc.). By another alternative, the physiological effect may be provision of a molecule or agent required for normal activity or metabolism, to cells lacking said molecule or agent or having said molecule or agent in very low levels. The physiological effect may be due to genetic manipulation of the genome of the cell, for genetic therapy purposes, for example for inducing cells to express within them an agent which is cytotoxic in order to destroy the cells themselves (for example, cancer cells) or for the purpose of inducing them to express an agent or molecule which the cells lack, for example due to a genetic disease, and which agent or molecule is required for normal activity. The physiological effect may be carried out at the cytosol in the membrane in the nucleus or in the extracellular space in the vicinity of the cell.

The term “target cell population” may concern cells of a single type for example erythrocytes. Alternatively, this term may concern cells of various types sharing some common feature such as cells of different types all carrying CD4 receptors. Typically, the target cell population, are cancer cells, cells infected by viruses such as HIV, hepatitis virus etc., cells of the immune system (for example for the treatment of autoimmune diseases), cells of infectious microorganisms or parasites which infiltrated the body and have to be destroyed, etc.

The prodrug of the invention comprises a complex between a nucleic acid molecule and a drug. Initially, the drug is in a physiologically inactive form, i.e. in a form which does not produce said physiological effect on said target cell population.

The nucleic acid molecule, is also initially in a catalytically inactive form, i.e., in a form which shows essentially no catalytic activity, or which has a very low catalytic activity.

Only where the, prodrug enters cells inside the target population, binds to cells of this population, or is in the vicinity of said cell population (thus being able to interact with agent secreted by the cells), does the nucleic acid molecule become catalytically active. The catalytic activity of the nucleic acid molecule, converts the drug from its physiologically inactive form, to its physiologically active form, thus producing the physiological effect on the said cell population.

The drug chosen for the prodrug is of course in accordance with the physiological effect which is desired.

The transition of the nucleic acid molecules from a catalytically inactive form, to a catalytically active form, preferably takes place inside cells of the desired cell population. The transition may take place due to special conditions present inside said cells of the population, such as pH, ionic strength, etc. However, preferably the transition of the nucleic acid molecule from the catalytically inactive to the catalytically active form takes place in the presence of an effector which may be an ion, a molecule, or a moiety within a molecule.

The effector, may be a molecule which is essentially present in all cells such as, for example, ATP in such a case, the prodrug itself is non-specific, and will induce its physiological effect in all cells to which it entered. Specificity, may be rendered by attaching to the prodrug a targeting moiety which is targeted only to the desired cell population. An example of such a targeting moiety is an antibody which ensures that the prodrug is introduced only into the target cell population. For example, the prodrug may be placed within a liposome, capable of entering cells by phagocytosis, and its targeting to the cells may be carried out by use of an appropriate antibody capable of recognizing only the target cell population, such as an antibody capable of specifically recognizing antigenic moieties specific to cancer cells. Another example of a targeting moiety is a ligand that binds only to cells carrying a specific receptor.

By another alternative, the nucleic acid molecules may transit from a catalytically inactive, to a catalytically active form, by an effector which effector is specific to the target cell population, i.e. an effector which is present in the target cell population, and which is essentially absent in non-target cell populations. Such a manner of transition of the nucleic acid molecule, ensures its specificity, so that the physiological effect of the active drug will be manifested only in the target cell population, and not in non-target cell populations.

The transition may take place in the cytosol of the cell, in its nucleus or at its membrane, after binding of the prodrug to receptors or components (such as proteins) present on the cells' surface. In the latter case, the receptors or components of the receptor may be the effectors. The transition may also take place in the extracellular space adjacent to cells of the population and in that case the effector may be a molecule or an agent (such as neurotransmitter) secreted from the cells of the desired population to the environment, and present in the extracellular space surrounding the desired cell population.

Example of effector molecules, which are present only in a target cell are viral proteins expressed in the cytosol of infected cells.

The manner in which the effector may activate the nucleic acid component of the drug is by an allosteric effect, for example according to one of the following.

Nucleic acid molecules, preferably RNA molecules, which become catalytically active only in the presence of effector molecules are known in the art. Porter, H. and Lizardi, P. M., “An allosteric hammerhead ribozyme” Bio/Technology, 13:101-104 (1995) and WO 94/13833 disclose ribozymes, being catalytic nucleic acid sequences, which become catalytically active in the presence of effector molecules. In the publication of Porter et al. the effectors are nucleic acid sequences and in WO 94/13833 the effectors may be either nucleic acid sequences or may be proteins. The transition of the ribozyme from a catalytically inactive form, to a catalytically active form induced by the effector, is effected by conformational change in the ribozymes. For example, in Porter et al., and WO 94/13833, the ribozyme contains an extra inhibitory sequence which folds in such a way so that it masks the catalytic core of the ribozyme rendering it inactive. When an effector molecule binds to said extra inhibitory sequence, it changes its conformation, thus unmasking the catalytic core of the ribozyme and converting it to its active form.

Another manner in which effector molecules can activate intially inactive nucleic acid sequences is specified in commonly owned International Application WO 98/08975. In this publication the nucleic acid sequence termed “protonucleozyme” is, a priori, catalytically inactive due to a lack of a component required for its catalytic activity and the effector molecule provides said component rendering the protonucleotzyme catalytically active. The missing component of the protonucleozyme may be completed by a non-nucleic acid effector, such as a protein, peptide, glycopeptide, hormone, etc. Alternatively, the missing component may be completed by a nucleic acid effector, and in such a case the missing component may be a missing segment of one or more nucleotides, or a missing bond between nucleotides.

Once the nucleic acid molecule becomes catalytically active, its catalytic activity converts the drug from its inactive form to its active form. This may be done, by any type of catalytic activity known to change a substrate, such as ligation, splicing out, splicing in, kinase activity, phosphorylation, esterification , cleavage etc. For example, the drug may be a nucleic acid sequence, (required for genetic therapy), which sequence in the inactive form of the drug is split into two parts. Only upon ligation of the two parts, by a catalytically active nucleic acid molecule, it becomes active in genetic therapy.

By a preferred embodiment, however, the drug is inactive since it is complexed, for example by covalent linking, to an inhibitory moiety which renders it inactive. The inhibitory moiety may cause this inhibition by masking the binding the region of the drug, or by sterically hindering the binding of the drug to its target. The inhibitory moiety may be the nucleic acid molecule itself, or may be an additional component of the prodrug associated with the drug and this additional component (which is neither the drug nor the potentially catalytic nucleic acid molecule) may be,a& nucleic acid or a non-nucleic acid molecule. If the drug in the prodrug of the invention is inactive due to the presence of the inhibitory moiety, then the actalytic activity of the nucleic acid sequence should be cleavage, which serves to remove the inhibitory moiety thus rendering the drug active. Preferably, the nucleic acid molecule of the prodrug of the invention is covalently linked, either directly or through said inhibitory moiety to the drug, and cis cleavage activity which detaches the nucleic acid sequences from the drug converts the drug from its inactive to its active form.

By a preferred embodiment of the invention, the cleavage between the drug and the inhibitory moiety, should be by cleavage of a non-naturally occurring bond or moiety. The advantage of using a non-naturally occurring bond or moiety in order to link the drug to its inhibitory moiety, is to ensure that disconnection between the drug and the inhibitory moiety, and thus activation of the drug, will not occur spontaneously, due to the presence of natural enzymes in the extracellular space, or inside cells and that activation of the drug will occur only due to catalytic activity of the nucleic acid sequence. For example, the drug may be bound to the inhibitory moiety through 2-methyl-RNA containing sequences. These non-naturally occurring nucleic acid sequence, is not recognized by any natural enzyme of the body. However, it is possible to tailor catalytic nucleic acid sequences, for example, through in vitro evolution, which are capable of cleavage of such a non-naturally occurring sequence.

The use of non-naturally occurring bonds or moieties renders the prodrug of the invention more specific, and safer for use than state of the art prodrugs, which are usually activated by innercellular enzymes naturally present in all cells, such as proteases.

By another aspect of the invention, termed “the binding aspect” the present invention concerns a complex between a drug capable of binding to a target associated with a cell or a biological tissue, and a nucleic acid sequence; wherein catalytic activity of the nucleic acid sequence serves to bind the drug to its target or to a moiety in the vicinity of its target or to strengthen a bond naturally produced between the drug, its target or a moiety in the vicinity of the target.

The purpose of this aspect, is to increase the affinity of a drug to its target or to increase the “effective concentration” of the drug in the vicinity its target as will be explained hereinbelow.

It is a well known fact, that many drugs are agonists, or antagonists of cellular receptors.

While the specificity of these drugs to their target receptors may be high, at times their Kd may be low, leading to dissociation of the drug from its receptor, and decreasing its physiological effect. Such a low Kd requires many times to increase the dose of the drug administered to the patient leading to severe side effects.

The complex of the present invention according to the binding aspect, is between a drug capable of binding to a target such as a receptor, an enzyme, etc. either for the purpose of activation of the target (agonists of a receptor, activator of an enzyme) or inactivation of the target (antagonists of a receptor, inhibition of the enzyme).

In the following the term “receptor” will be used at times to replace the term “target” although it should be understood that the target may be moieties other than receptors such as for example enzymes.

In order to increase the Kd of the drug to its target receptor, it is possible to use a catalytic nucleic acid complexed to the drug, which, can cause covalent binding of the drug either to its receptor, or to a moiety or molecule in the vicinity of the receptor. Where the drug is bound directly to the receptor this ensures the drug will not dissociate at all from the receptor. Where the drug is bound to a molecule or moiety in the vicinity of the receptor this ensures that even if the drug dissocates from its receptor, it stays in the vicinity of the receptor and is thus capable of re-association, thus increasing significantly the “effective concentration” of the drug in the region close to the receptor. Thus although circulating concentrates of the drug may be low, concentration near its target remains high.

The cataltyic nucleic acid molecule should bind the drug to the receptor, by the following types of catalytic activities: ligation, phosphrilation, esterfication, creation of a peptidic bond etc.

The nucleic acid molecule may be a priori active and for example the target is the substrate of the catalytic activity of the sequence. Only when the drug binds to its target moiety, for example, its receptor, does the nucleic acid molecule come into contact with its substrate, so that binding between the drug and the receptor occurs.

By another alternative, the nucleic acid molecule may be initially inactive, and becomes catalytically active by an effector in the manner described in detail in the “drug aspect” of the invention. The effector, in that case may be a molecule or a moiety within a molecule associated either with the target (the receptor) or associated with cells or tissue which carry the receptor. For example, the effector, may be a soluble protein present or secreted by cells carrying the target of the invention.

The present invention will now be illustrated with reference to some non limiting examples.

### BRIEF DESCRIPTION OF THE DRAWINGS

FIGS. 1A to 1D shows the prodrug of the invention;

FIGS. 2A to 2J shows the manner of preparation of the prodrug of the invention by in vitro evolution;

FIGS. 3A to 3C shows a complex between a drug and an a priori active nucleic acid sequence; and

FIGS. 4A to 4C shows a complex between a drug and an a priori inactive nucleic acid sequence which is activated by an effector.

### DETAILED DESCRIPTION OF A PREFERRED EMBODIMENT

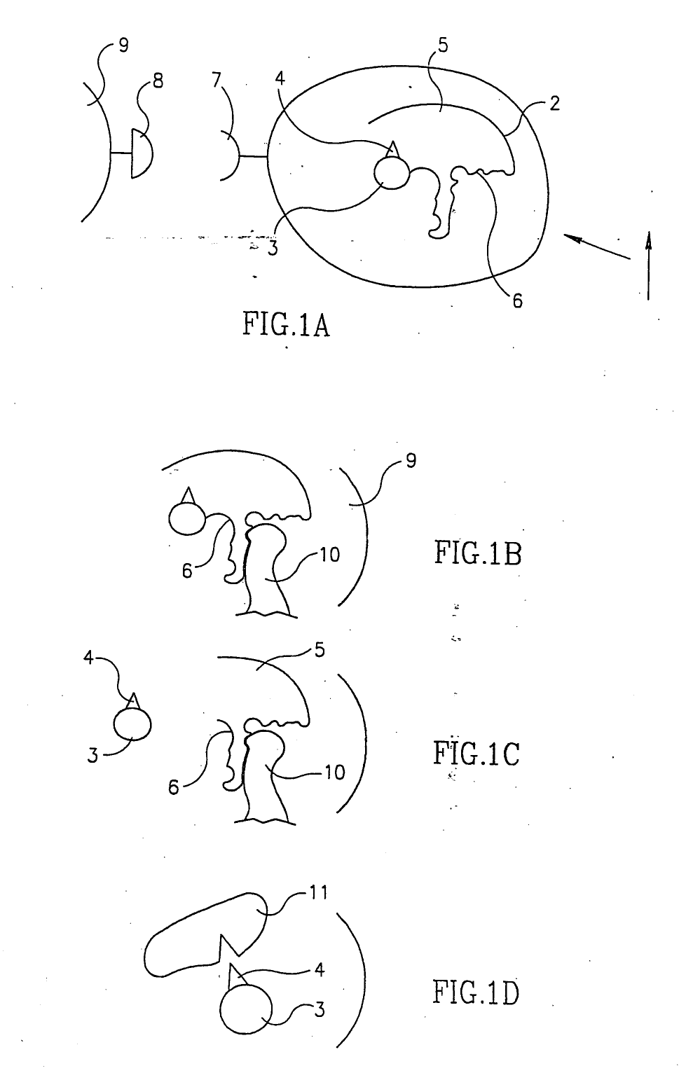

FIG. 1 shows the manner of activity of the prodrug of the invention. FIG. 1A shows a lipsome 1 encapsulating the prodrug of the invention 2. The prodrug is composed of a drug 3 having an active site 4 which is masked by an inhibitory moiety 5, rendering the drug 3 inactive. The inhibitory moiety 5, is linked to the drug through nucleic acid molecule 6 which is inactive due to lack of a missing component. The liposome carries a targeting moiety 7 being an antibody capable of specifically binding to receptor 8 of cell 9 of the target cell population. Receptor 8, is specific to the target cell population. The drug, for example is a cytotoxic drug. In FIG. 1B, the lipsome entered the cell by a phagocytosis, and the membrane of the liposome was digested by cellular enzymes, thus exposing the prodrug to the cells' cytosol. Whilst exposed, effector molecule 10, for example being a protein, can bind to the nucleic acid molecule 6, completing its missing component and transferring nucleic acid molecule 6 from its inactive to its catalytically active form.

The effector molecule 10 may be specific to the target cell population, for example a protein present only in the target cell population such as viral protein characterizing viral infected cells, or alternatively, may be general to all cells. In the latter case specificity of the prodrug as a whole is rendered only by targeting means 7 which ensures specific entry only to the cell of the population through receptor 8 which is specific for the population.

FIG. 1C, shows that once the nucleic acid molecule is active, it cleaves itself in cis from the drug 3, thus removing both itself and removing the inhibitory moiety 5 from the drug, and exposing active site 4.

The cleavage between nucleic acid molecule 6 and drug 3, may be by cleavage of a non-naturally occurring bond, or of a non-naturally occurring moiety, such as 2-methyl-RNA sequence, thus ensuring that no naturally occurring enzyme present in cell 9, (such as DNases, RNases, etc.) will be able to convert the drug spontaneously to its active form.

Once active drug 3 is free from the inhibitory moiety 5, its active site 4 can bind to an appropriate component 11 in the cell, carrying out the desired physiological effect, such as cytotoxic effect, and thus destroying the cell.

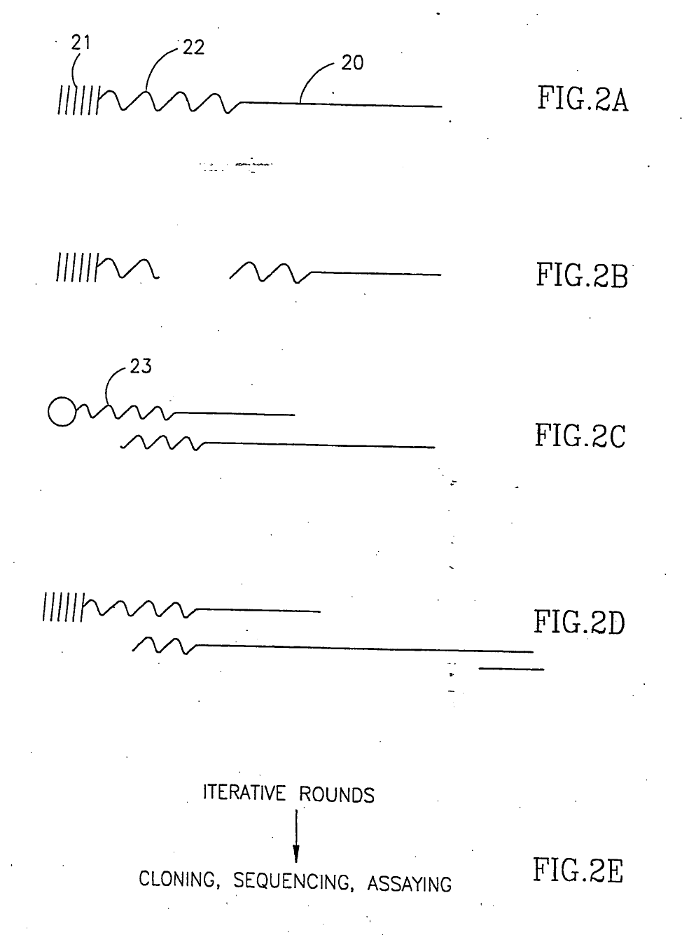

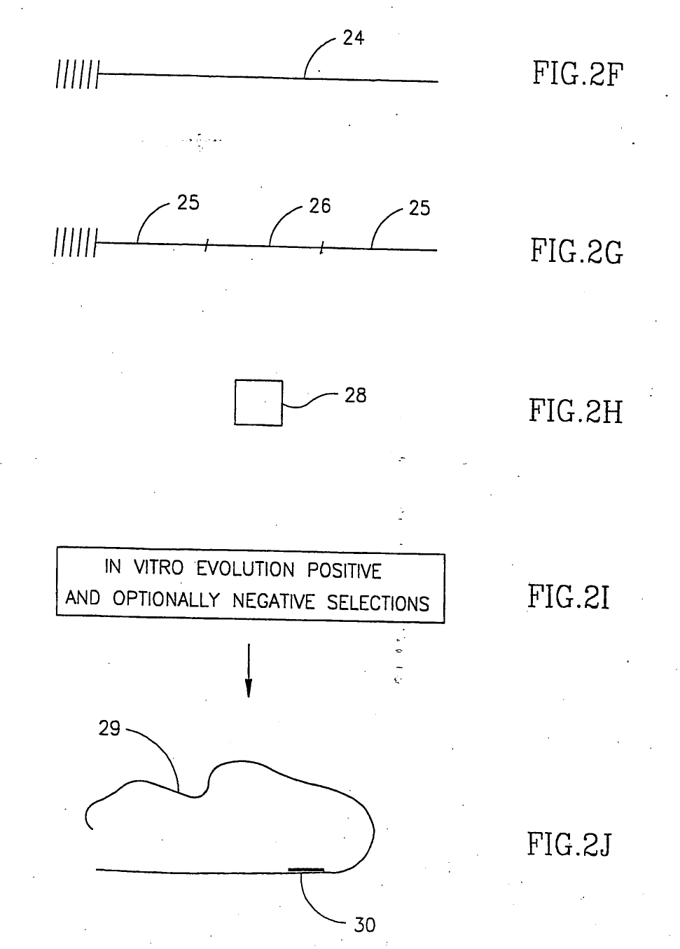

FIG. 2 shows the manner of production of the prodrug shown in FIG. 1 utilizing in vitro evolution.

FIG. 2A shows the first step of selection during in vitro evolution. A population of random sequences 20, are prepared with a 2′-omethyl substrate 22, in a manner where they are bound to a solid support 21. The ribozyme-solid support constructs are allowed to react in physiological conditions. In FIG. 2B sequences are then chosen which have been cleaved from the solid support within the 2-omethyl region, for example as shown in FIG. 2C by using a probe 23 complimentary to part of the sequence that is comprised of the 2′-o-methyl.

Following selection the pool is amplified via a PCR using primers from the flanking sequences and having the 2′-o-methyl sequence reconstructed onto the 5′ end of one of the PCR primers (FIG. 2D). This sequence is subjected iterative rounds of selection and amplification, after which, the final round is cloned, sequenced and assayed for activity (FIG. 2E). Then the ribozyme can be made dependent on the effector molecule. The product 24 produced (FIG. 2E) is “doped” (i.e. a certain percentage of its nucleotides are made random) in that a pool is made that has a fixed amount of mutations, the doped regions are denoted as 25. New randomized regions 26 can also be introduced to produce a sequence 27 comprising both “doped” regions 25 and random sequence 26 (FIG. 2G). This sequence is now brought into contact with an effector 28, being in fact identical to molecule 10 in FIG. 1 (FIG. 2H). Only ribozymes which feature catalytic activity in the presence of the effector are freed and collected. If desired, a negative selection step or several negative selection steps may be introduced wherein ribozymes featuring catalytic activity in the absence of the correct effector molecule are removed (FIG. 2I). The pool is subjected to in vitro evolution similar to the that mentioned above, and the best ribozymes are cloned, sequenced, and assayed for activity. In step (2J), randomized sequences developed through in vitro evolution in a similar manner as disclosed above are developed and selected for those sequences 29 which are capable of folding in such a manner that they mask a drug 30 present thereon and thus rendering the drug inactive. In such a manner only cleavage is cis frees drug 30 from the complex. This final step is thus suitable for producing the prodrug of the invention.

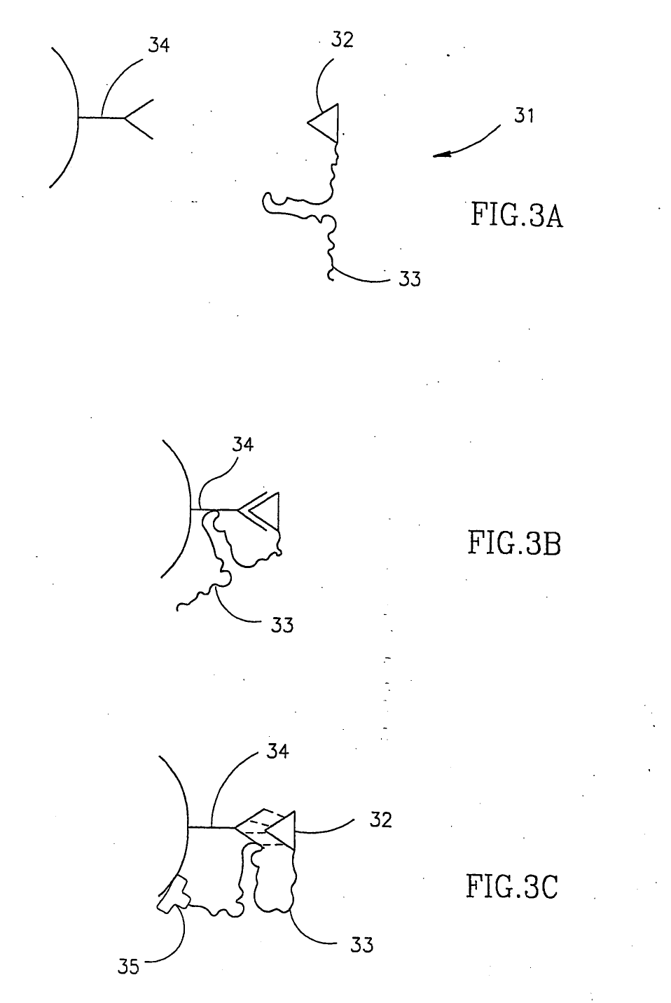

FIG. 3, shows the binding aspect of the invention. The complex of the invention 31 is composed of a drug 32, linked to a catalytically active nucleic acid molecule 33. The drug is capable of binding to a target moiety, for example to a receptor 34.

Once the drug binds to the receptor 34, the catalytic nucleic acid sequence, through the catalytic activity of creation of a peptidic bond, links the complex covalently to the receptor, so that it is covalently bound to the receptor as shown in FIG. 3B, thus increasing the Kd of the drug to its receptor.

By another alternative shown in FIG. 3C, the nucleic acid molecule 33 binds to a moiety in the vicinity of the receptor 35, for example, a protenaceous component of the membrane, or another subunit of the receptor. In such a manner, although the complex is not directly linked to the receptor to which it should bind, drug 32, stays always in close vicinity to receptor 34, and is capable of easy re-association, they increase the “effective concentration” of the drug in its vicinity.

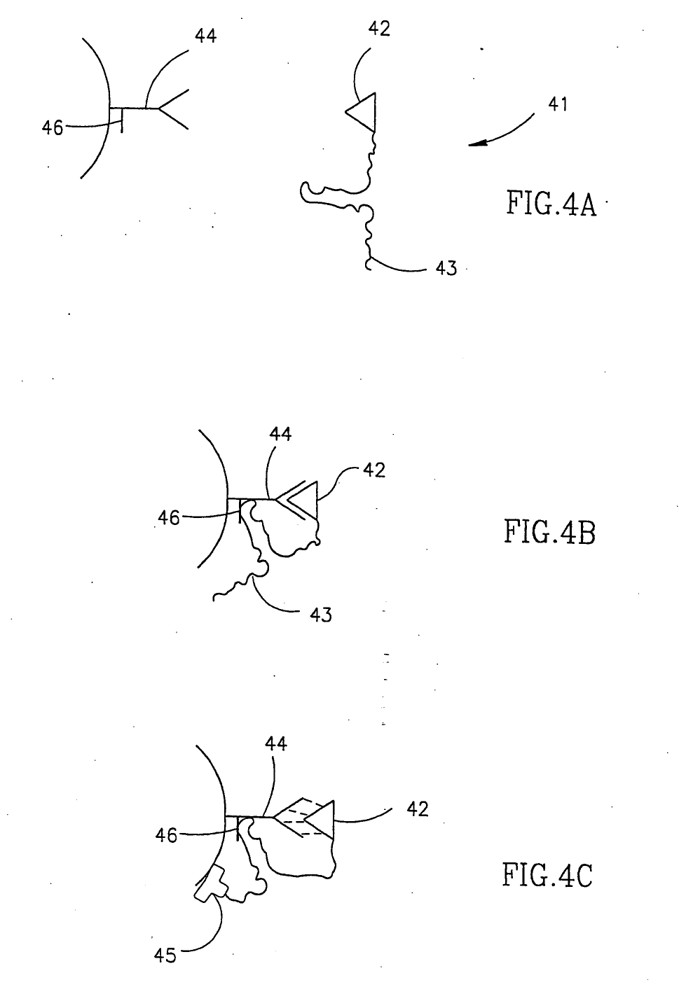

FIG. 4A shows essentially the same complex shown in FIG. 3, termed 41.

However, while in FIG. 3, the ribozyme 33, was a priori active, and all that is was required to exert its catalytic activity, was the correct substrate, being either receptor 34 or moiety 35, in FIG. 4A, the nucleic acid molecule 43 is a priori inactive due to reasons specified above (presence of an inhibitory-masking moiety or lack of an essential component)

When drug 42 binds to its correct receptor 44, then nucleic acid molecule 43 comes into contact with its effector 46, which for example as shown in the figure may be a moiety within receptor 44. Effector 46, activates nucleic acid molecule 43, for example by causing a conformational change, or by completing a missing component as explained above.

In FIG. 4B, said activation, leads to the binding of the ribozyme to the receptor, covalently linking the prodrug to its receptor.

In FIG. 4C, the activation of the ribozyme by effector moiety 46 leads to its binding to moiety 45, which is in the vicinity of the receptor 44, for example of subunit of this receptor, ensuring that even if the prodrug disassociates from the receptor, its effective concentration in the vicinity of the receptor remains quite high.

1. A prodrug for use in inducing a physiological effect in a target cell population comprising a drug which can be converted from an inactive form, in which it does not induce said physiological effect, to an active form in which it induces said physiological effect, the prodrug comprising the drug in its inactive form and being characterized by:
the prodrug comprises a complex between a nucleic acid molecule and said drug in the inactive form; the nucleic acid molecule being capable of transition from a catalytically inactive form to a catalytically active form in which catalytically active form the nucleic acid molecule causes said drug to convert to the physiologically active form; said transition of the nucleic acid molecule is dependent on the presence of an effector being a nucleic acid sequence.

2. A prodrug according to claim 1 wherein the effector is present in the target cell population and is essentially absent in the non-target cell populations.

3. A prodrug according to claim 1 or 2 wherein the physiological effect is a cytotoxic effect.

4. A prodrug according to claim 1 or 2 wherein the catalytic activity of the nucleic acid sequence is cleavage in cis within the complex.

5. A prodrug according to claim 4 wherein the cleavage is of a non-naturally occurring bond, or cleavage within a non-naturally occurring moiety.

6. A prodrug according to claim 1 or 2 wherein the drug and the nucleic acid molecule are covalently bound to each other.

7. A prodrug according to claim 1 or 2 wherein the nucleic acid molecule is essentially an RNA sequence.

8. A prodrug according to claim 1 or 2, wherein the effector causes a conformational change in the nucleic acid molecule thereby causing its transition from its catalytically inactive form to its catalytically active form.

9. A prodrug according to claim 1 or 2 wherein the effector provides a component missing from the nucleic acid molecule thereby rendering it active.

10. A prodrug according to claim 9 wherein said missing component is a missing segment of one or more nucleotides or a missing bond between two nucleotides.

11. A prodrug according to claim 1 or 2 wherein the target cell population are cancer cells.

12. A prodrug according to claim 1 or 2 wherein the target cell population are cells infected by viruses.

13. A prodrug according to claim 1 or 2 further comprising a targeting moiety capable of targeting the prodrug to the target cell population.

14. A prodrug according to claim 13 wherein the targeting moiety is an antibody.

15. A complex between a drug, capable of binding to a target associated with a cell or a biological tissue, and a nucleic acid sequence; wherein the catalytic activity of the nucleic acid sequence results in binding the drug to its target or to a moiety in the vicinity of its target.

16. A complex according to claim 15 wherein the catalytic activity is selected from the group consisting of: ligation, phosphrilation, esterification, creation of a peptidic bond.

17. A complex according to claim 15 or 16 wherein the binding is between the nucleic acid sequence and the target or a moiety in the vicinity of the target.

18. A complex according to claim 15 wherein the target is a receptor.

19. A complex according to claim 15 or 16 wherein the nucleic acid sequence is a priori catalytically active.

20. A complex according to claim 15 or 16, wherein the nucleic acid sequence is a priori inactive but becomes catalytically active by an effector which is a molecule or moiety within a molecule that is specific to the target that is specific to a moiety in the vicinity of the target or that is specific to the cells or tissues associated with the target.
